(19) United States
(12) Patent Application Publication
(10) Pub. No.: US 2005/0256572 A1
(43) Pub. Date: Nov. 17, 2005

(54) Breast prosthesis

(76) Inventor: Helmut Wild, Rosenheim (DE)

Correspondence Address: ALLEMAN HALL MCCOY RUSSELL & TUTTLE LLP, 806 SW BROADWAY, SUITE 600, PORTLAND, OR, 97205-3335, US

(21) Appl. No.: 11/131,444

(22) Filed: May 16, 2005

(30) Foreign Application Priority Data
May 17, 2004 (DE) 20 2004 007 921.6

(51) Int. Cl. A61F 2/52

(52) U.S. Cl. 623/7; 623/926

(57) ABSTRACT

The present application relates to a breast prosthesis having bodies welded in plastic films and modeled on the shape of the human breast, the prosthesis being made at least partially of a silicone rubber mass or of another soft-elastic material. In accordance with one embodiment, at least some of the plastic films comprise gas-tight material(s).

### CROSS-REFERENCE TO RELATED APPLICATION

This application claims priority to German Patent Application Serial No. DE 202004007921.6 filed May 17, 2005, which is hereby incorporated by reference in its entirety for all purposes.

### FIELD

The present application relates to a breast prosthesis.

### BACKGROUND AND SUMMARY

A method is known, for example from DE 27 01 627 A 1, of manufacturing breast prostheses from shell-shaped bodies welded into plastic films, modeled on the shape of the breast and from an addition cross-linked two-component silicone rubber mass. The prostheses manufactured with this method are almost ideal copies of the natural breast in their appearance and in their behavior due to the elastic softness, the movement, the consistence and the weight of the material used.

The breast prostheses are attached to the breast of the wearer in a manner as nonslipping as possible. For this purpose, it is known, for example from EP 392960 A 1, to provide breast prostheses of the type first named with a peripheral shoulder formed by a step on its rear side inside a peripheral lip-shaped rim, with adhesive strips or adhesive pieces being fastened to said peripheral shoulder which cooperate with the adhesive regions of strips fastened to the body of the woman by skinfriendly adhesive means such that the prosthesis is connected to the holding strips adhering to the body and can be released from them again. A hook and loop connection is, for example, provided as the fastening means.

Breast prostheses, to the extent that they are not designed to be self-adhesive or in connection with adhesive strips, are usually worn in special brassieres with an integrated pouch or also partly directly on the skin, which can result in unpleasant heat accumulation with the formation of perspiration due to the restricted exchange of air beneath the prosthesis and the poor heat transport.

Prostheses are also already known which are made in two layers and in which an inner side facing the breast has a chamber which is filled with a liquid, preferably a thixotropically set liquid. When such a breast prosthesis is worn, an unwanted formation of blisters occurs to a greater extent due to the aforesaid formation of heat accumulation and due to a corresponding discharge of perspiration in the region of the liquid. This mainly occurs due to the interaction between the plastic film usually consisting of polyurethane and the liquid which does not have a fixed connection to the film and to the mechanical deformation of the film and the liquid during wear. This phenomenon is amplified by the increased humidity which develops between the breast prosthesis and the body. The formation of blisters in particular occurs with a mixture of thixotropic liquids with light fillers in the part of the two-layer prosthesis facing the body.

It has furthermore been observed with breast prostheses that visible blisters occur close to the prosthesis surface. In particular with silicones which are provided with light fillers, such as has become increasingly usual over the past few years, but also with prosthesis chambers which are filled with liquid, a tendency toward blister formation has become increasingly visible when the prosthesis is subject to large air pressure fluctuations, e.g. due to local altitude differences.

Such a blister formation is due to the fact that silicones tend to absorb gases, and thus also air, up to the respective saturation limit and in dependence on the environmental pressure. This can be compared with an absorption capability of a sponge. On a change in the ambient pressure, the absorbed gas volume again attempts to adapt in accordance with the environmental pressure, which can result in clearly visible gas blisters close to the prosthesis surface on an air pressure reduction. To prevent this beading of the gas in the finished prosthesis, the silicones are “evacuated” before the processing to ensure that no more air is located in the material. It can nevertheless be observed that corresponding air accumulates after some time in the breast prostheses even if they were absolutely air-free directly after the manufacture, that is if no blister formation was able to be observed. This air accumulation with possible later blister formation is in particular observed in the customary breast prostheses in which the plastic films consist of polyurethane films when they are exposed to larger fluctuations in air pressure, for example due to the altitude of the location of use.

It is one object of the present application to further develop generic breast prostheses such that a blister formation inside the breast prosthesis is reduced.

This object may be solved by a breast prosthesis comprising bodies welded in plastic films, modeled on the shape of the breast and made of a silicone rubber mass or of another soft-elastic material, where at least some of the plastic films comprise gas-tight material. Accordingly, no air or water vapor can penetrate into the silicone at the corresponding interface due to the created gas barrier. An exchange of air resulting in blisters can thus also not occur on changes in air pressure.

In one embodiment, the breast prosthesis can have a plastic film as a special form of a gastight plastic film which is impermeable to water vapor or which inhibits water vapor at least at its side facing the body. Films with a water vapor block can therefore be used here. This is in particular of advantage with multi-layer prostheses, preferably two-layer prostheses, with here—in accordance with an embodiment variant—only the layer close to the body being surrounded at least partly by a plastic film impermeable to water vapor. At least the film coming into contact with the body should here be impermeable to water vapor. In accordance with an alternative embodiment, however, the whole layer close to the body can also be enveloped by means of the plastic film impermeable to water vapor or inhibiting water vapor in the two-layer prosthesis.

Another gas-tight embodiment variant consists of the fact that the breast prosthesis is at least partly air-tight, i.e., that an air-tight plastic film has been used here.

The gas-tight plastic films can consist at least partly of one or more of the following components:
ethylene vinyl alcohol (EVOH), polyvinylidene chloride (PVDC), polyvinyl alcohol (PVAL), polyamides (PA), highly drawn polypropylene (PP), propylene oxide (HPPO), polyethylene naphtha late (PEN).

Nano-composites can also advantageously be added to the raw materials of the gas-tight plastic films. Furthermore, the gas-tight plastic films can support a plasma coating with SiOx or be evaporation coated with aluminum. The materials can also be combined with other plastics, for example with polyethylene (PE), for the gas-tight plastic film.

The materials for a plastic film impermeable to water vapor can consist at least in part of plastic blends such as a polyurethane blend with polyethylene, polypropylene or other polyolefins and/or blends of pure polyolefins. It is particularly advantageous to use the plastic film impermeable to water vapor in two-layer prostheses where the second layer close to the body contains a thixotropic liquid, very advantageously a thixotropic liquid with light fillers.

### BRIEF DESCRIPTION OF THE FIGURES

Further features and details and advantages will be explained in more detail with reference to an embodiment shown in the drawing. There are shown:

FIG. 1: a longitudinal section through a breast prosthesis in accordance with a first embodiment; and

FIG. 2: a longitudinal section through a breast prosthesis in accordance with a second embodiment.

### DETAILED DESCRIPTION

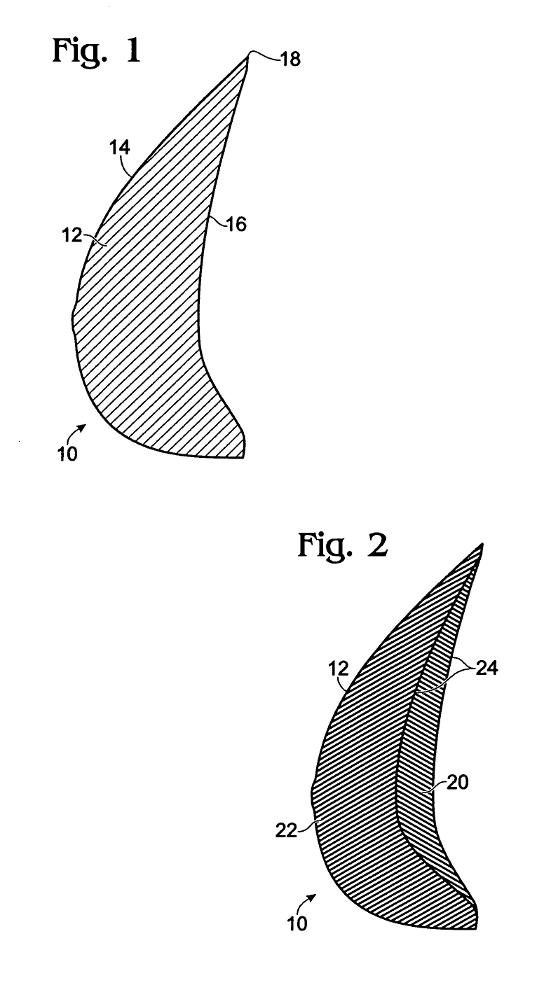

The breast prosthesis 10 shown in FIG. 1 includes a shell-shaped body 12 made of a soft-elastically set addition cross-linked two-component silicone rubber mass whose outside is covered by a plastic film 14 and whose inside is covered by a plastic film 16 which are connected to one another along a common peripheral rim 18 by a peripheral welding seam.

In the embodiment shown here, the plastic films 16 and 14 each consist of a plastic which represents a gas barrier and thus does not permit a saturation of the silicone with air or at least makes it considerably more difficult.

The embodiment in accordance with FIG. 2 shows a breast prosthesis 10 as a two-layer prosthesis with a layer 20 close to the body and a layer 22 remote from the body. The outer layer 22 remote from the body consists of conventional addition cross-linked two-component silicone rubber, that is of a standard silicone. It can alternatively also consist of a so-called light silicone, that is of an addition cross-liked two-component silicone rubber, to which a component is mixed which lowers the specific gravity of the mass. The inner layer 22 consists of a thixotropic liquid, for example a non-linked silicone oil in which light fillers have been embedded. The layer 20 close to the body is welded into a plastic film 24 which consists of a material impermeable to water vapor. In the embodiment shown here, the water vapor block is formed, for example, by polyurethane blends with polyethylene, polypropylene or other polyolefins. In contrast, the outer layer 22 remote from the body is surrounded by a plastic film 12 of conventional polyurethane. The breast prosthesis 10 in accordance with FIG. 2 admittedly does not have the gas tightness in accordance with that of FIG. 1, but the blister formation is securely prevented in the region of the layer 20 close to the body or is made substantially more difficult in that the plastic film 24 has a water vapor block. A penetration of water vapor into the region of the thixotropic liquid which is mixed with the light fillers is therefore securely prevented.

In accordance with a further embodiment variant not shown in any more detail here, the polyurethane layer 12 could also be replaced by a plastic film which is impermeable to gas.

1. A breast prosthesis comprising bodies welded in plastic films, said films modeled on a shape of a breast and made of a soft-elastic material, where at least some of the plastic films comprise gas-tight material.

2. A breast prosthesis in accordance with claim 1, where the plastic film is impermeable to water vapor at least on its side facing a body wearing the prosthesis.

3. A breast prosthesis in accordance with claim 2, wherein the prosthesis is a multi-layer prosthesis, and wherein only a layer close to the body is surrounded at least in part by a plastic foil impermeable to water vapor.

4. A breast prosthesis in accordance with claim 1, wherein the prosthesis is at least partly air-tight.

5. A breast prosthesis in accordance with claim 1, wherein the gas-tight plastic film comprise at least in part at least one material of: ethylene vinyl alcohol (EVOH), polyvinylidene chloride (PVDC), polyvinyl alcohol (PVAL), polyamides (PA), highly drawn polypropylene (PP), propylene oxide (HPPO), and polyethylene naphthalate (PEN).

6. A breast prosthesis in accordance with claim 5, wherein nano-composites or liquid crystalline polymers (LCPs) have been added to the raw materials of the plastic films.

7. A breast prosthesis in accordance with claim 1, wherein the gas-tight plastic films support a plasma coating with SiOx.

8. A breast prosthesis in accordance with claim 5, wherein said at least one material for the gas-tight plastic film is combined with another plastic.

9. A breast prosthesis in accordance with claim 1, wherein the material for a plastic film impermeable to water vapor comprises at least in part plastic blends.

10. A breast prosthesis in accordance with claim 1, wherein a thixotropic liquid is contained in a second layer of a two-layer prosthesis close to a body wearing the prosthesis.

11. A breast prosthesis in accordance with claim 1, wherein the gas-tight plastic films have been evaporation coated with aluminum.

12. A breast prosthesis in accordance with claim 8, wherein said another plastic is polyethylene (PE).

13. A breast prosthesis in accordance with claim 9, wherein said plastic blends include at least one of a polyurethane blend with polyethylene, polypropylene or other polyolefins.

14. A breast prosthesis in accordance with claim 9, wherein said plastic blends include pure polyolefins.

15. A breast prosthesis in accordance with claim 1, wherein said prosthesis is made of a silicone rubber mass.

16. A breast prosthesis in accordance with claim 3, wherein the prosthesis is made as a two-layer prosthesis.

17. A breast prosthesis, comprising:
a body including silicone welded in a plastic film, said film modeled on a shape of a breast and made of a soft-elastic material, where at least a portion of the plastic film comprises a gas-tight material which reduces a saturation of the silicone with air even during variation is ambient pressure.

18. A breast prosthesis, comprising:
a first layer; and
a second layer configured to be located on a side coupled to a body of a user wearing the prosthesis, the second layer having a body including silicone welded in a plastic film, said film modeled on a shape of a breast and made of a soft-elastic material, where at least a portion of the plastic film comprises a gas-tight material which reduces a saturation of the silicone with air even during variation is ambient pressure.
